(19) United States
(12) Patent Application Publication
(10) Pub. No.: US 2018/0329108 A1
(43) Pub. Date: Nov. 15, 2018

(54) MOBILE MULTI-MODALITY PASSIVE SENSING PLATFORM FOR PASSIVE DETECTION OF RADIOLOGICAL AND CHEMICAL/BIOLOGICAL MATERIALS

(71) Applicant: TETRA TECH MIMDU, LLC, Pasadena, CA (US)

(72) Inventors: John Peter Gonsky, JR., Richland, WA (US); Matthew David Neuman, Newtown, PA (US)

(73) Assignee: TETRA TECH MIMDU, LLC, Pasadena, CA (US)

(21) Appl. No.: 15/824,738

(22) Filed: Nov. 28, 2017

Related U.S. Application Data
(60) Provisional application No. 62/504,921, filed on May 11, 2017.

(51) Int. Cl. G01V 5/00; G01T 1/20; G01N 33/00; G01S 19/01

(52) U.S. Cl. CPC G01V 5/0075; G01T 1/20; G01W 2001/006; G01S 19/01; G01N 33/0004

(57) ABSTRACT

A mobile system for the in situ detection of radiological hazards and mass identification of volatile organic and inorganic compounds, and biological agents using completely passive detection technology provided in multiple subsystems. Data from the subsystems is sent to a graphical user interface and the information from all applicable subsystems is displayed on a single user interface.

### CROSS-REFERENCE TO RELATED APPLICATION

This application claims priority to U.S. Provisional Patent Application Ser. No. 62/504,921 for a “Mobile Multi-Modality Passive Sensing Platform for Passive Detection of Radiological and Chemical/Biological Materials” filed on May 11, 2017, the contents of which are incorporated herein by reference in its entirety.

### FIELD

This disclosure relates to the field of detection of potentially harmful radiological and chemical substances. More particularly, this disclosure relates to the passive detection of radiological and chemical substances on a moving or otherwise mobile platform.

### BACKGROUND

Illicit materials affect authorities all over the globe. Radioactive materials and explosive compounds can be used individually or in tandem to create widespread havoc on an area. Increasingly, terrorist organizations are relying on triacetone triperoxide (TATP) and diacetone triperoxide (DATP) as a means for local destruction, and the movement of radioactive material on the black market could lead to weapons of mass destruction being provided to organizations with the intent to do harm. Combining these two hazards can lead to radiological dispersion devices, or “dirty” bombs, where initial chaos is created with a downstream fallout from radioactive materials. In addition, weaponized chemical and biological agents are some of the worst weapons of mass destruction materials.

The illegal drug market is getting bigger and the smugglers are getting more creative with their methods. The rate of illegal drugs passing through secured U.S. borders is high. Manufacture of illegal drugs is also a concern inside the U.S. and many other countries.

Environmental monitoring is becoming a larger issue as the effects of environmental contaminants are becoming more prevalent. Un-tracked environmental discharges can have local and global effects on the population, from poor air quality, to global warming.

Explosive materials are generally identified by K-9 (dog) teams. Dogs can detect the presence of groups of explosive chemicals, but are unable to specifically identify the chemical. Dogs, however, have a useful working time of only a few hours per day. Dogs must be housed, fed, and trained. Dogs must be replaced every few years. Other non-dog explosive detectors are available to screen people crossing thru a checkpoint, but are limited to a handheld device screening one person at a time. These detectors do not provide real time data on the type or relative quantity of material. In addition, the handheld detectors are often fooled by other legitimate materials, such as some colognes and makeup products.

Detection of illicit or smuggled radioactive material is sophisticated and well developed. Most detector systems are either large fixed systems or small hand-held detectors. In hand held applications, either gross radiation detectors or heavy radioisotope identification systems are used, but they must always be close to the target.

Drugs are generally identified by K-9 (dog) teams. Additionally, field reconnaissance is often used to determine smuggling routes, manufacturer areas, or higher level dealers. Increasingly the more sophisticated synthetic drugs are now proving to be fatal to dogs when inhaled.

Environmental monitoring is typically performed with large scientific instruments. The usual concept of operations includes taking a sample (air, water, soil) and transporting it to the location of the instrumentation for analysis. Real time in situ monitoring and analysis for broad-spectrum detection of environmental contaminants is not well established.

Individual dogs can be trained only to detect specific contraband thereby requiring different dogs for different illicit material searches. Given the wide range and ever-changing range of illicit materials encountered, the demand for different types of dogs in any given situation can never be met. Furthermore, biological agents are not readily detected, even by dogs

Real-time in situ identification of the chemical, biological, radiological, nuclear, and explosive (CBRNE) materials described above would provide a significant tool in the global fight against terror, the drug war, and environmental sustainability. Existing solutions that can perform real time identification of all of these illicit materials in the described problems are few. There are no known fully passive detector systems that can address all of these needs in one mobile unit.

What is needed, therefore, is a mobile vehicle capable of passively detecting a broad range of CBRNE materials including radiological detection and chemical substance detection.

### SUMMARY

The above and other needs are met by a mobile platform for the real-time in situ detection of radiological hazards and mass identification of volatile organic and inorganic compounds, and biological agents using completely passive detection technology. The mobile system incorporates both radioactive material detection and airborne chemical/biological material detection equipment integrated by a single operating, data analysis, and data archiving system with a single user interface. The onboard radiation, chemical, biological, and explosives systems can detect, identify, and in some cases quantify illicit materials within fractions of a second, making the system detection capability nearly instantaneous. This rapid detection and identification permits activation of alarms. Unlike prior art systems, all detector subsystems are fully passive, not emitting any form of radiation or energy. The integrated interface system includes a command and control capability, allowing an operator to direct security forces or hazardous material response personnel to a location of detected illicit material as it is being detected. Combined archived system data can be used to prosecute individuals caught performing illegal acts.

Catastrophic incidents can be avoided by deploying this system to various high traffic areas such as seaports, land ports of entry, transportation hubs, public facilities, schools, stadium facilities, and airports that may be targeted for radiological dispersal devices, conventional explosive devices, or biological agents. Additionally, the system can be used to develop an environmental baseline of chemical contaminant conditions. Because of the mobile nature of the system, it can then detect and track the movement of the contaminants in real time in situations of chemical pollution accidents or intentional releases. In addition, the system can be used to detect and interdict smuggled explosives, drugs, or radioactive materials.

In a preferred embodiment, a mobile multi-modality passive sensing system includes a mobile vehicle; a passive radiological detection device mounted in the mobile vehicle for detecting potentially harmful radiological materials proximate to the mobile vehicle; a passive chemical detection device mounted in the mobile vehicle for detecting potentially harmful chemical materials proximate to the mobile vehicle; a user interface device located in the mobile vehicle; a central processing device located in the mobile vehicle wherein the central processing device is in communication with the passive radiological detection device, the passive chemical detection device, and the user interface device, and wherein the central processing device (i) receives data from (1) the passive radiological detection device related to potentially harmful radiological materials proximate to the mobile vehicle and (2) the passive chemical detection device related to potentially harmful chemical materials proximate to the mobile vehicle; (ii) processes the data resulting with processed data; and (iii) sends the processed data to the user interface device to be displayed on the user interface device. Preferably, the system further comprises an air sampling subsystem comprising a tube connected to the chemical detection device and a vacuum pump for drawing in air wherein air is drawn into the tube from outside the mobile vehicle and directed to the chemical detection device for real time analysis.

In yet another embodiment, a mobile multi-modality passive sensing system includes a mobile vehicle; a passive radiological detection device mounted in the mobile vehicle for detecting potentially harmful radiological materials proximate to the mobile vehicle; a passive chemical detection device mounted in the mobile vehicle for detecting potentially harmful chemical materials proximate to the mobile vehicle; and a computing device comprising a processor and a user interface device, the computing device located in the mobile vehicle in communication with the radiological detection device and the chemical detection device wherein data from the radiological detection device and the chemical detection device is sent to the computing device for processing and then to the user interface device to be displayed to a user.

The mobile multi-modality passive sensing system preferably further comprises an air sampling subsystem comprising a tube connected to the chemical detection device and a vacuum pump for drawing in air wherein air is drawn into the tube from outside the mobile vehicle and directed to the chemical detection device for real time analysis.

The mobile multi-modality passive sensing system may further include a weather substation in communication with the computing device for gathering weather data and relaying such weather data to the computing device.

Additionally or alternatively, the mobile multi-modality passive sensing system may further include a GPS device in communication with the computing device for providing GPS location data to the computing device.

The mobile multi-modality passive sensing system may further include a computer readable storage device for archiving data originating from the radiological detection device, the chemical detection device, and the weather substation and/or the GPS device.

The mobile vehicle of the mobile multi-modality passive sensing system further including a window and at least one camera directed toward the window for collecting images gathered through the window, and wherein the at least one camera is in communication with the computing device.

The mobile multi-modality passive sensing system may further include a command and control communications subsystem including a handheld detector, a vest detector, a backpack detector, one or more additional mobile vehicles, or any combinations thereof.

A method of passively monitoring an environment for potential chemical dangers and potential radiological dangers using a mobile vehicle is also disclosed, the method comprising the steps of (i) operating a passive radiological detection device on a mobile vehicle to acquire radiological data; (ii) operating a passive chemical detection device on the mobile vehicle to acquire chemical data; (iii) sending the radiological data to a computing device; (iv) sending the chemical data to the computing device; (v) processing the radiological data; (vi) processing the chemical data; and (vii) sending the processed radiological data and the processed chemical data to a display device for displaying the processed radiological data together with the processed chemical data. The method may further include the step of producing an alarm condition signal to be displayed on the display device if the radiological data or the chemical data exceed a minimum concentration threshold. The method may further include the step of archiving the radiological data if the radiological data exceed a minimum concentration threshold and/or archiving the chemical data if the chemical data exceed a minimum concentration threshold. The method may further include the step of tagging the radiological data and/or the chemical data with GPS coordinates of the mobile vehicle in response to the alarm condition signal. The method may further include the step of archiving video data gathered from a camera mounted on the mobile vehicle in response to the alarm condition signal.

The summary provided herein is intended to provide examples of particular disclosed embodiments and is not intended to cover all potential embodiments or combinations of embodiments. Therefore, this summary is not intended to limit the scope of the invention disclosure in any way, a function which is reserved for the appended claims.

### BRIEF DESCRIPTION OF THE DRAWINGS

Further features, aspects, and advantages of the present disclosure will become better understood by reference to the following detailed description, appended claims, and accompanying figures, wherein elements are not to scale so as to more clearly show the details, wherein like reference numbers indicate like elements throughout the several views, and wherein:

FIG. 1 shows a schematic drawing of a mobile multi-modality passive sensing system including multiple subsystems according to one embodiment of the present disclosure;

FIG. 2 shows a schematic side view of a mobile sensing system according to one embodiment of the present disclosure;

FIGS. 3 and 4 show a radiological subsystem according to one embodiment of the present disclosure;

FIG. 5 shows a front view of a radar sensor according to one embodiment of the present disclosure;

FIG. 6 shows a side view of a radar sensor according to one embodiment of the present disclosure;

FIG. 7 shows a top plan view of a mobile sensing system according to one embodiment of the present disclosure;

FIG. 8 shows side view of a mobile sensing system according to one embodiment of the present disclosure;

FIG. 9 shows a schematic of an air sampling subsystem according to one embodiment of the present disclosure;

FIG. 10 shows a circuit schematic of a mobile sensing system according to one embodiment of the present disclosure;

FIG. 11 shows a diagram of a computing device according to one embodiment of the present disclosure;

FIG. 12 shows a diagram of a sensing system according to one embodiment of the present disclosure;

FIGS. 13-15 illustrate a graphical user interface according to one embodiment of the present disclosure; and

FIG. 16 shows a command and control system according to one embodiment of the present disclosure.

The figure(s) is/are provided to illustrate concepts of the invention disclosure and is/are not intended to embody all potential embodiments of the invention. Therefore, the figure(s) is/are not intended to limit the scope of the invention disclosure in any way, a function which is reserved for the appended claims.

### DETAILED DESCRIPTION

Various terms used herein are intended to have particular meanings. Some of these terms are defined herein for the purpose of clarity. The definitions given herein are meant to cover all forms of the words being defined (e.g., singular, plural, present tense, past tense). If the definition of any term herein diverges from the commonly understood and/or dictionary definition of such term, the definitions herein control.

“Air” is defined as any gas or mixture of gases including any particulate solids and liquids entrained therein.

“Real time” is defined as an actual time during which a process or event occurs or within a minimum time lapse of a minimum number of minutes from such process or event occurring such as 30 minutes, more preferably 10 minutes, even more preferably less than five minutes, and most preferably less than one minute.

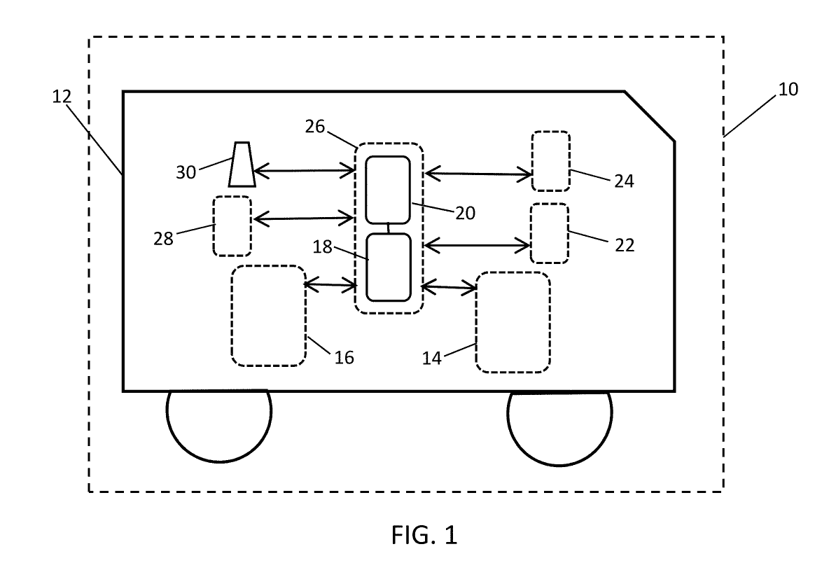

FIG. 1 shows a schematic drawing showing a mobile multi-modality passive sensing system 10 including a mobile vehicle 12 for passive detection of radiological and chemical/biological materials. The mobile multi-modality passive sensing system 10 allows for the detection of nearby illicit materials to positively identify a chemical compound or radionuclide. Embodiments of the system enable generation of an alarm condition, such as an alarm that identifies a chemical compound or radioactive material and a relative intensity of that material.

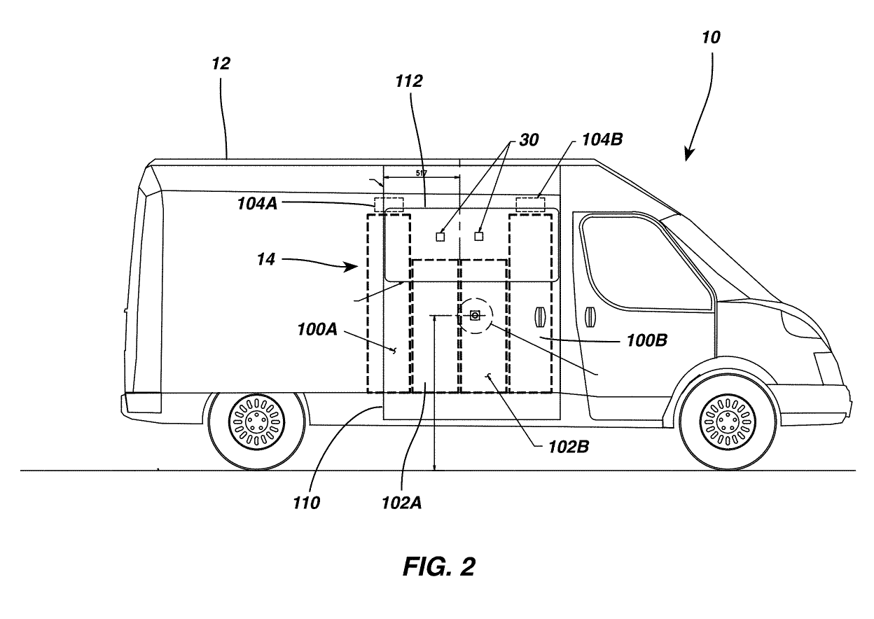

The mobile vehicle 12 is preferably in the form of a van (FIG. 2) that houses a plurality of purely passive detectors including a radiological materials detector 14 (or “radiation subsystem”) and a chemical/biological materials detector 16 (or “chemical/biological subsystem”). Additional passive detectors can be included in related embodiments. The at least two detectors are in direct and/or wireless communication with a central processing device 18 which is in further communication with a user interface device 20 such as, for example, a touch screen device or computer display and keyboard. The radiological materials detector 14 and the chemical/biological materials detector 16 are preferably operated simultaneously, each sending output data regarding detected materials to the central processing device 18 where the data is processed and sent to the user interface device 20 in a viewable format so that data from the at least two detectors can be visually monitored simultaneously from the same user interface by a person operating the user interface device 20.

The radiation subsystem 14 detects and identifies radioactive and nuclear material. The chemical/biological subsystem 16 detects and identifies commonplace and unique airborne chemical, biological, and explosive compounds. Both subsystems identify and trigger an alarm within seconds of detection of a potentially threatening radiological and/or chemical/biological material close to the mobile vehicle 12. The overall passive sensing system 10 preferably includes seven main subsystems including the mobile vehicle 12, the radiation subsystem 14, the chemical/biological subsystem 16 (e.g., a proton transfer reaction mass spectrometer), an air sampling subsystem 22, an electrical subsystem 24, a user interface and operations subsystem 26 (including the central processing device 18 and the user interface device 20, providing data acquisition and archiving capability), and a command and control communications subsystem 28. All of these subsystems are preferably integrated together into a seamless mobile passive sensing system 10 that can be programmed to detect specific types or sources of radiation, or specific chemical/biological compounds that correlate to specific hazards. The various subsystems are networked together to electronically communicate with one another. The system 10 can be used in either mobile or stationary operation, as described below.

The mobile platform or mobile vehicle 12 used as a part of the overall system 10 has few constraints. The platform (vehicle 12) is adequate provided that the other (preferably six) subsystems can be comfortably installed and operate inside the vehicle 12. For most uses, the subsystem equipment will be installed in automobiles or vans, but can also be installed in planes/helicopters, boats, or trains. Subsystem equipment may be installed in the mobile vehicle 12 according to embodiments described below.

### Radiation Detector Subsystem

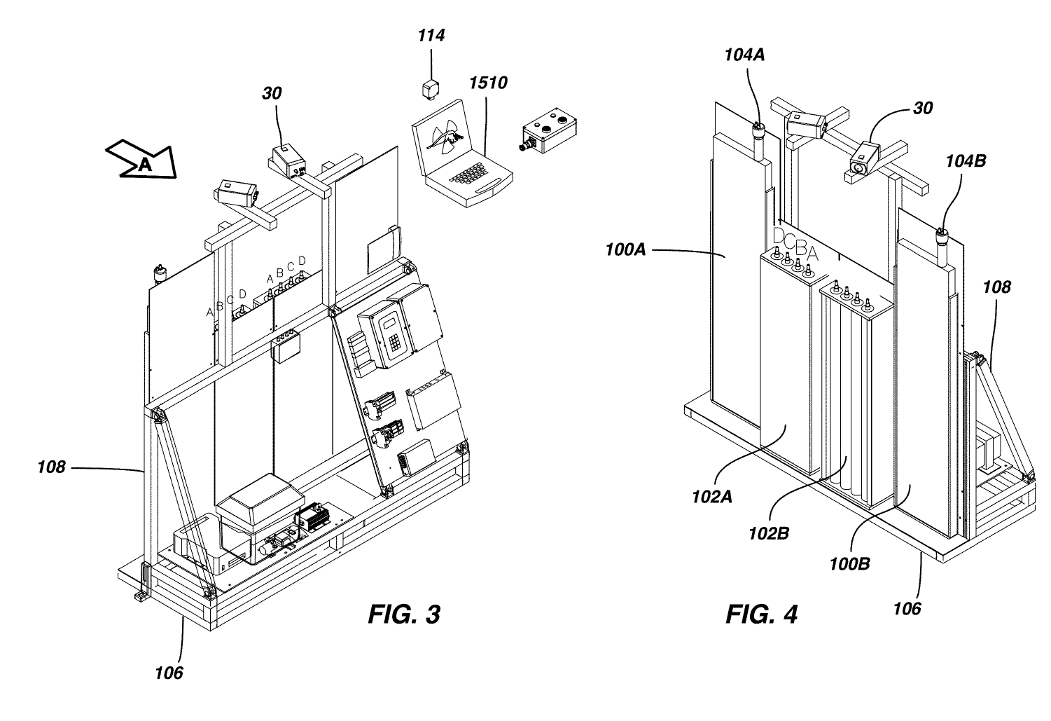

Referring to FIGS. 2-4, the radiation detection subsystem 14 preferably includes two types of scintillating gamma detectors 100A and 100B as well as neutron detectors 102A and 102B. The gamma detectors 100A and 100B are preferably comprised of large area polyvinyl toluene (PVT) plastic detectors and may include crystalline sodium iodide (NaI) detectors 104A and 104B located at an upper portion of the gamma detectors 100A and 100B. The large area PVT detectors preferably include a surface area that maximize an ability of the gamma detectors 100A and 100B to detect the presence of nuclear materials within proximity of the radiation detection subsystem 14.

The gamma detectors 100A and 100B are utilized to detect the presence of gross gamma radiation, while the NaI detectors 104A and 104B may be used to identify the specific isotopic source of the radiation. The neutron detectors 102A and 102B are preferably formed of several groups of Boron-10 (B-10) proportional counters for detecting neutrons.

The radiation subsystem 14 is mounted on a radiation subsystem skid 106 within the mobile vehicle 12. The radiation subsystem skid 106 includes a plurality of frame members 108 for supporting the radiation detection subsystem 14 within the mobile vehicle 12. The radiation detection subsystem 14 is further mounted on the radiation subsystem skid 106 such that the radiation detection subsystem 14 may be installed or removed as a module, thereby enabling removal of the radiation subsystem 14 for repair or replacement of components of the radiation detection subsystem 14. As described above and as shown in FIGS. 2-4, the radiation detection subsystem 14 preferably includes at least a pair of the gamma detectors 100A and 100B and neutron detectors 102A and 102B to reduce intrinsic error in the radiation detection subsystem 14. Data sent from the radiation subsystem 14 can include, for example, neutron count rate, gamma count rate, radioisotopic identification and/or radionuclide family identification (e.g., naturally occurring radiation, medical radiation, industrial radiation, or special nuclear material).

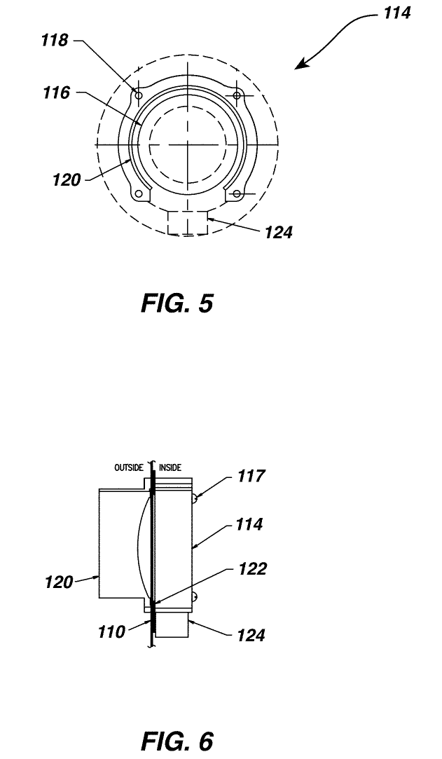

Referring again to FIG. 2, when the mobile vehicle 12 is a van, the radiation subsystem 14 is preferably mounted adjacent to and aligned with a door 110 of the mobile vehicle 12. The door 110 preferably includes a window 112 formed through the door 110. In one embodiment, the radiation subsystem 14 further includes a radar sensor 114 mounted on the door 110 adjacent to the radiation subsystem 14. Referring now to FIGS. 5 and 6, the radar sensor 114 is preferably configured to detect a presence of an object, such as a person, vehicle, or other object, within a proximity of the radar sensor 114. The radar sensor 114 is preferably mounted through a cutout 116 the door 110 and is secured to the door with one or more fasteners 117 installed through a bracket 118 of the radar sensor 114. The radar sensor 114 also includes a weather shield 120 formed at least partially around a portion of the radar sensor 114 disposed outside of the door 110 for protecting the radar sensor 114 from outside elements. The radar sensor 114 may further include a gasket 122 located between the radar sensor 114 and the door 110 to further seal the radar sensor 114 from the elements. As shown in FIG. 6, the radar sensor 114 includes a connector 124 located on an inside portion of the door 110 for communication of the radar sensor 114 with other components of the passive sensing system 10 as described in greater detail below. While the above description contemplates a radar sensor 114 for detecting presence of an object, other various motion detectors or sensors may be suitable for detecting the presence of an object within proximity of the mobile vehicle 12.

### Chemical/Biological Subsystem

The chemical/biological detector subsystem 16 preferably includes a proton transfer reaction mass spectrometer (PTRMS) 125 that may be used to identify airborne chemical (e.g. explosive, drug, or environmental) and biological compounds by continuously measuring a molecular mass of the compounds from air drawn thru the air sampling subsystem 22 system from outside the vehicle 12 in real time. The mass is then correlated with known substances with unique mass numbers. The sampled medium is the air surrounding the vehicle 12 drawn into and thru the mass spectrometer by the air sampling subsystem 22. The chemical/biological subsystem 16 is configured to detect volatile organic compounds (VOCs) of interest to help identify a presence of illicit materials of interest.

In one embodiment, the PTRMS 125 of the chemical/biological detector subsystem 16 is an existing available instrument, such as a model TOF1000-Ultra available from Ionicon. The chemical/biological detector 125 preferably has a sensitivity of greater than 400 cps/ppbv and a resolution of greater than 1,500. The chemical/biological detector subsystem 16 is capable of performing real-time organic trace gas analysis and allows for a quantitative analysis of the entire mass range in real-time and can detect specific masses of interest when searching for illicit materials. The chemical/biological detector subsystem 16 further records raw data for masses being detected to provide a chemical background signature of an area of interest. Data from the chemical/biological detector subsystem 16 can include, for example, molecular mass of the principal components of the detected compound(s) and/or relative intensities of the components.

The chemical/biological detector subsystem 16 is preferably configured to detect triacetone triperoxide (TATP) that may be used as an explosive. The chemical/biological detector subsystem 16 preferably detects TATP by detecting its trace VOC molecules emitted. The chemical/biological detector subsystem 16 receives air from the air sampling subsystem as described in greater detail below for analysis and identification of illicit materials in an area of interest.

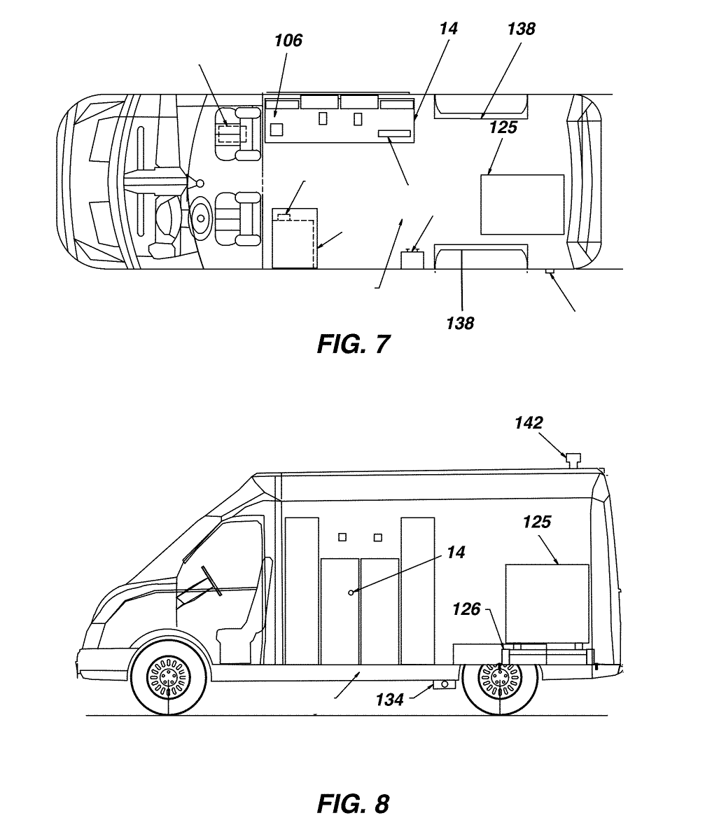

Referring now to FIGS. 7 and 8, the chemical/biological detector 125 is preferably mounted inside of the mobile vehicle 12, such as in a rear cargo area of the mobile vehicle 12. The chemical/biological detector 125 is preferably mounted to a chemical detector skid 126. The chemical detector skid 126 is preferably mounted to the mobile vehicle 12, and may be removable from the mobile vehicle 12 such that the chemical/biological detector subsystem 16 is substantially modular. The chemical detector skid 126 may further include one or more shock absorbers for protecting the chemical/biological detector subsystem 16 during movement of the mobile vehicle 12.

### Air Sampling Subsystem

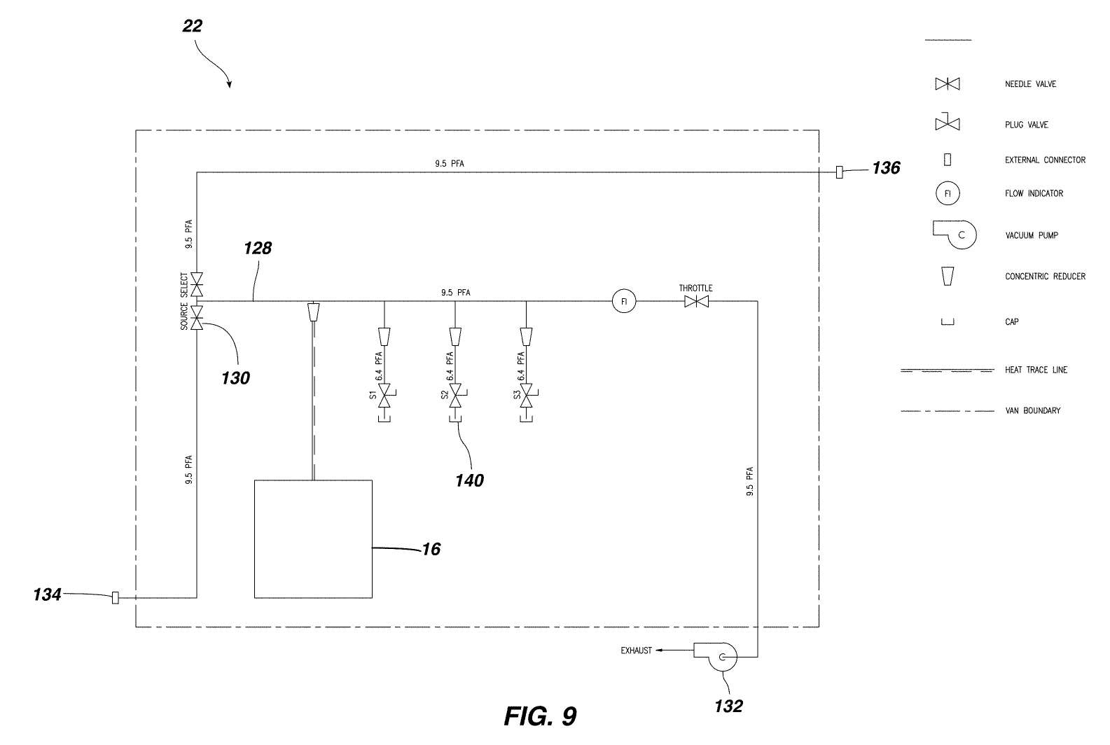

The air sampling subsystem 22 (FIG. 1) allows the overall system 10 to draw a continuous flow of air surrounding the vehicle 12 and provide that air to the inlet of the proton transfer reaction mass spectrometer 125. Referring to FIG. 9, the air sampling subsystem 22 includes a series of tubes 128, valves 130, and a vacuum pump 132 that can be manipulated to draw air from multiple ports around the vehicle 12 and through one or both of a driver side intake 134 and a passenger side intake 136. Additionally, the air sampling subsystem 22 can utilize a sampling hose to draw air from within a container, vehicle, or from a greater distance from the vehicle 12. This allows for unique sample locations as opposed to general area samples.

Referring to FIG. 9, the air sampling subsystem 22 is in fluid communication with the proton transfer reaction mass spectrometer 125 of the chemical/biological detector subsystem 16 such that air drawn into the air sampling subsystem 22 through one or both of the driver side intake 134 and passenger side intake 136 flows to the chemical/biological detector subsystem 16 for analysis. The valves 130 may be arranged such that intake air is selectively received from either the drive side intake 134 or the passenger side intake 136, or both.

The tubes 128 are preferably formed of ⅛″ perfluoroalkoxy (PFA) tubing and appropriate PFA fittings. The driver side air intake 134 and passenger side air intake 136 are preferably located adjacent fenders 138 of the mobile vehicle and at a desired height above the ground, such as between 12″ and 24″ from the ground surface. The valves 130 are preferably needle valves, however other known valves may be used in the air sampling subsystem 122. The vacuum pump 132 is preferably a diaphragm vacuum/compressor pump that is mounted on an underside of the mobile vehicle 12.

The air sampling subsystem 22 is configured to provide a flow rate of between 5 std. L/min and 10 std. L/min. Flow rate may be controlled by the user interface device 20. The drive side intake 134 and passenger side intake 136 are adapted to receive an extension hose such that air may be sampled from a location that is remote to a location of the mobile vehicle 12. The air sampling subsystem 22 may include capped auxiliary tubes 140 that are capable of receiving and being connected to additional environmental monitoring equipment for further analysis of air received in the air sampling subsystem 22.

The system 10 may further include a weather substation 142 (FIG. 8) located on the mobile vehicle 12. The weather substation 142 may be selected from known weather sensors, and preferably includes sensors for detecting a speed and direction of wind relative to the mobile vehicle 12, and may include additional sensors for collecting data related to other atmospheric conditions of the mobile vehicle 12 and surrounding areas. A suitable weather station 142 may comprise, for example, a model WS-220WX weather station from Airmar. The weather station preferably measures apparent wind, true wind, barometric pressure, air temperature, wind chill temperature, dew point temperature, and heat index temperature.

### Electrical Subsystem

The electrical subsystem 24 is designed to provide auxiliary power to the radiation subsystem 14, the chemical/biological subsystem 16, the air sampling subsystem 22, the user interface and operations subsystem 26, and the command and control communications subsystem 28. The electrical subsystem 24 allows the rest of the overall system 10 to run independently from external power sources. The electrical subsystem 24 can also utilize external power sources to provide power for extended periods of time. The internal electrical subsystem either uses a small generator or power from the vehicle alternator, depending upon the vehicle make and model selected as the mobile platform. Use of one or more batteries is a backup if other power sources are not available. Power transformers and conditioners are provided for those nations where other than 60 Hz 120 volt power is standard.

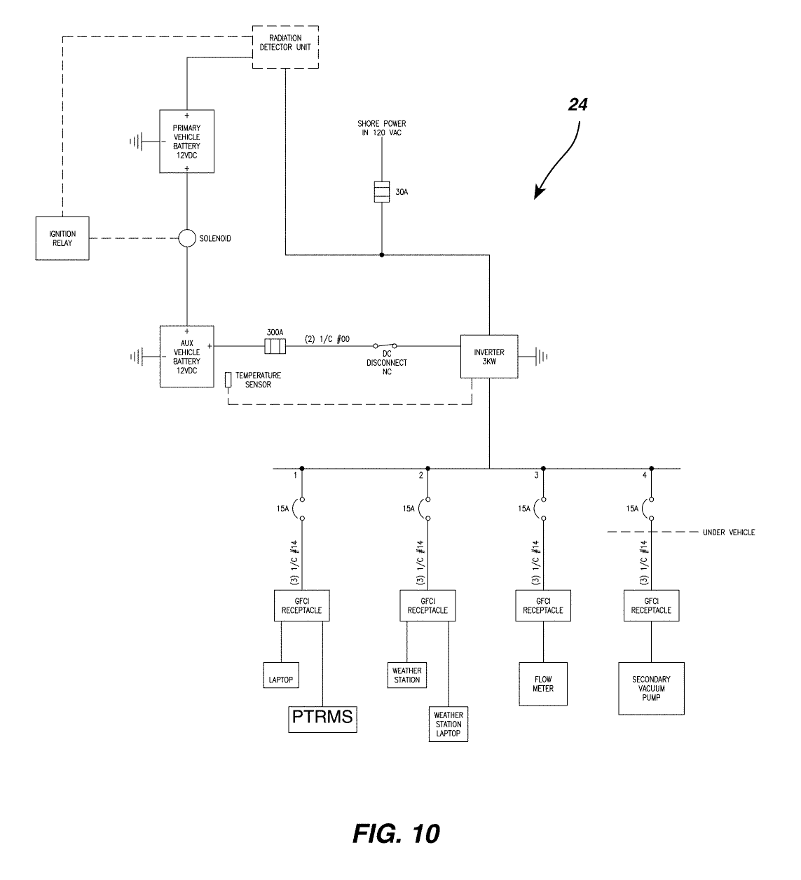

Referring to FIG. 10, the electrical subsystem 24 is adapted to provide power to subsystems of the mobile multi-modality passive sensing system 10 and may include one or more auxiliary batteries for supplying power to the subsystems separate from a battery and electrical system of the mobile vehicle 12. The one or more auxiliary batteries may be arranged such that an alternator of the mobile vehicle 12 provides charging to the one or more auxiliary batteries while the mobile vehicle 12 is running.

### User Interface and Operations Subsystem

The user interface and operations subsystem 26 provides a graphical user interface (i.e., via the user interface device 20) for the operation of the overall system 10. The user interface and operations subsystem 26 including the central processing device 18 is designed to consolidate data output from the radiation subsystem 14, chemical/biological subsystem 16, one or more video cameras 30 in communication with the central processing device 18, and meteorological data provided from an external source or from onboard meteorological instrumentation 142 in communication with the central processing device 18. The system 10 can archive data to a data storage device described below wherein the data can be used later to prosecute those caught in the act of smuggling or transporting illicit materials. The data acquisition subsystem includes features for monitoring of local weather patterns and recording video of vehicles or people causing the system to alarm. The system is configured to alarm for specific compounds of interest and provide video playback of the alarm events. The weather information is used to help identify the direction of the origin of the chemical source material.

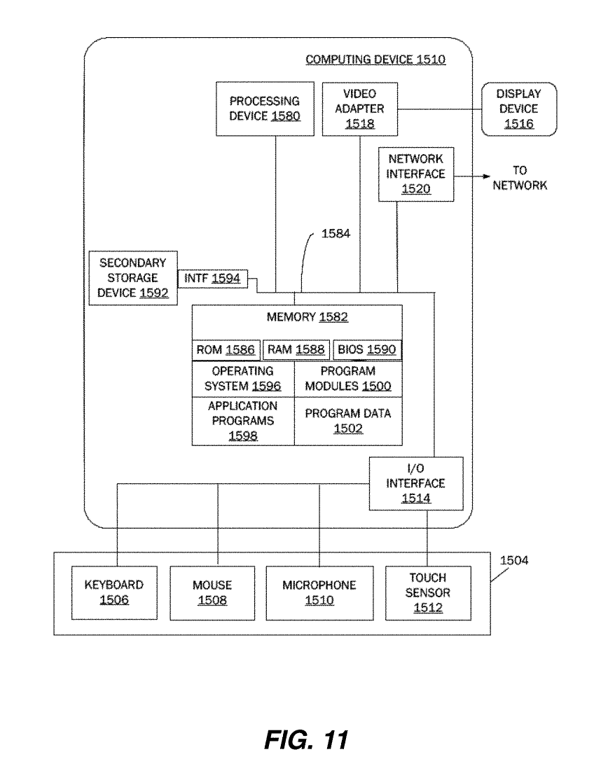

Referring to FIG. 11, the user interface and operations subsystem 26 is preferably operably on and includes a computing device 1510. The computing device 1510 includes at least one processing device 1580, such as a central processing unit (CPU) 18. A variety of processing devices are available from a variety of manufacturers, for example, Intel or Advanced Micro Devices. In this example, the computing device 1510 also includes a system memory 1582, and a system bus 1584 that couples various system components including the system memory 1582 to the processing device 1580. The system bus 1584 is one of any number of types of bus structures including a memory bus, or memory controller; a peripheral bus; and a local bus using any of a variety of bus architectures.

Examples of computing devices suitable for the computing device 1510 include a desktop computer, a laptop computer, a tablet computer, a mobile computing device (such as a smart phone, a tablet device, or other mobile devices), or other devices configured to process digital instructions.

The system memory 1582 includes read only memory 1586 and random-access memory 1588. A basic input/output system 1590 containing the basic routines that act to transfer information within computing device 1510, such as during start up, is typically stored in the read only memory 1586.

The computing device 1510 also includes a secondary storage device 1592 in some embodiments, such as a hard disk drive, for storing digital data. The secondary storage device 1592 is connected to the system bus 1584 by a secondary storage interface 1594. The secondary storage devices 1592 and their associated computer readable media provide nonvolatile storage of computer readable instructions (including application programs and program modules), data structures, and other data for the computing device 1510.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media. Additionally, such computer readable storage media can include local storage or remote or cloud-based storage including, for example, one or more servers.

A number of program modules can be stored in secondary storage device 1592 or memory 1582, including an operating system 1596, one or more application programs 1598, other program modules 1500 (such as the software engines described herein), and program data 1502. The computing device 1510 can utilize any suitable operating system, such as Microsoft Windows™, Google Chrome™, Apple OS, and any other operating system suitable for a computing device. Other examples can include Microsoft, Google, or Apple operating systems, or any other suitable operating system used in tablet computing devices. Preferably, a proprietary operating system is used.

In some embodiments, a user provides inputs to the computing device 1510 through one or more input devices 1504 which can form part of the user interface device 20. Examples of input devices 1504 include a keyboard 1506, mouse 1508, microphone 1510, and touch sensor 1512 (such as a touchpad or touch sensitive display). Other embodiments include other input devices 1504. The input devices are often connected to the processing device 1580 through an input/output interface 1514 that is coupled to the system bus 1584. These input devices 1504 can be connected by any number of input/output interfaces, such as a parallel port, serial port, game port, or a universal serial bus. Wireless communication between input devices and the interface 1514 is possible as well, and includes infrared, BLUETOOTH® wireless technology, 802.11a/b/g/n, cellular, or other radio frequency communication systems in some possible embodiments.

In this example embodiment, a display device 1516, such as a monitor, liquid crystal display device, projector, or touch sensitive display device, is also connected to the system bus 1584 via an interface, such as a video adapter 1518. Such display device 1516 can form part of the user interface device 20. In addition to the display device 1516, the computing device 1510 can include various other peripheral devices (not shown), such as speakers or a printer.

When used in a local area networking environment or a wide area networking environment (such as the Internet), the computing device 1510 is typically connected to a network through a network interface 1520, such as an Ethernet interface. Other possible embodiments use other communication devices. For example, some embodiments of the computing device 1510 include a modem for communicating across the network.

The computing device 1510 typically includes at least some form of computer readable media. Computer readable media includes any available media that can be accessed by the computing device 1510. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 1510.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 11 is also an example of programmable electronics, which may include one or more such computing devices, and when multiple computing devices are included, such computing devices can be coupled together with a suitable data communication network so as to collectively perform the various functions, methods, or operations disclosed herein.

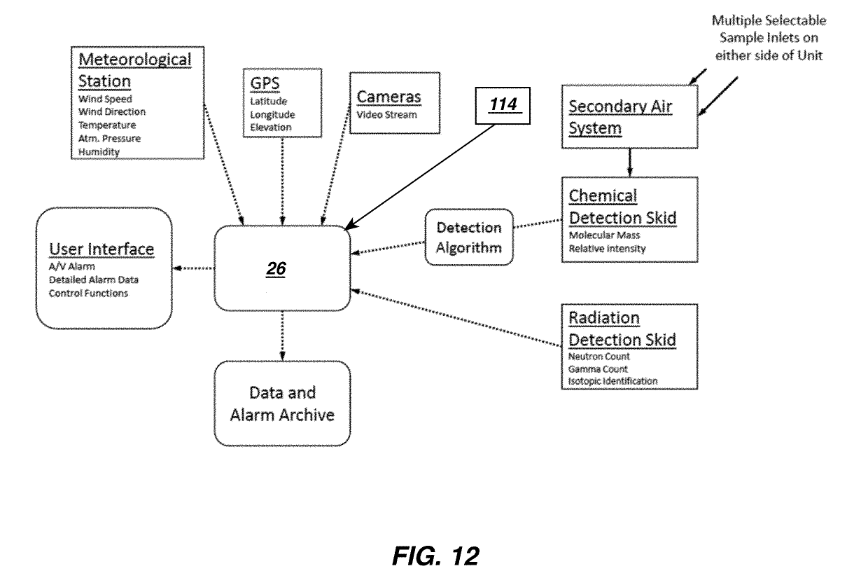

Software operable on the computing device 1510 enables data captured from the various subsystems described above to be centrally analyzed and to provide system status, operating controls, and alarm information to an operator. Data analyzed by the user interface and operations subsystem 26 provides whether an alarm condition exists based on data analyzed by the computing device 1510, including both chemical and radiological alarms based on data captured and analyzed from the radiation subsystem 14 and the chemical/biological subsystem 16. As shown in FIG. 12, data from multiple subsystems is centrally analyzed to identify potentially hazardous threats based on the data measured by the subsystems. Some preprocessing of the data is necessary in order to determine the nature of the contaminants detected. The operating system software compares the data received from the detector systems to libraries of known illicit materials in order to positively identify the chemical compound or radionuclide. The computing device 1510 also makes real time decisions, comparing measured concentrations (intensities) or activities of chemical compounds and radioactive substances to known background concentrations.

As for the chemical/biological subsystem 16 for example, relative concentrations of molecular mass numbers are normalized to account for atmospheric variability. Analysis is then performed on the normalized data to determine whether the measured concentration of airborne contaminants are statistically significant above background and electronic noise levels. If statistically significant (such that there is high confidence of the presence of a contaminant), then an alarm condition signal is generated. Further, a concentration analysis may be used to generate a scaled alarm condition signal depending on a threat identified by the system 10. Data related to the molecular mass detected by the system 10 may be compared to a library of threat compounds (such as explosives, drugs, and environmental contaminants) to identify potentially illicit compounds.

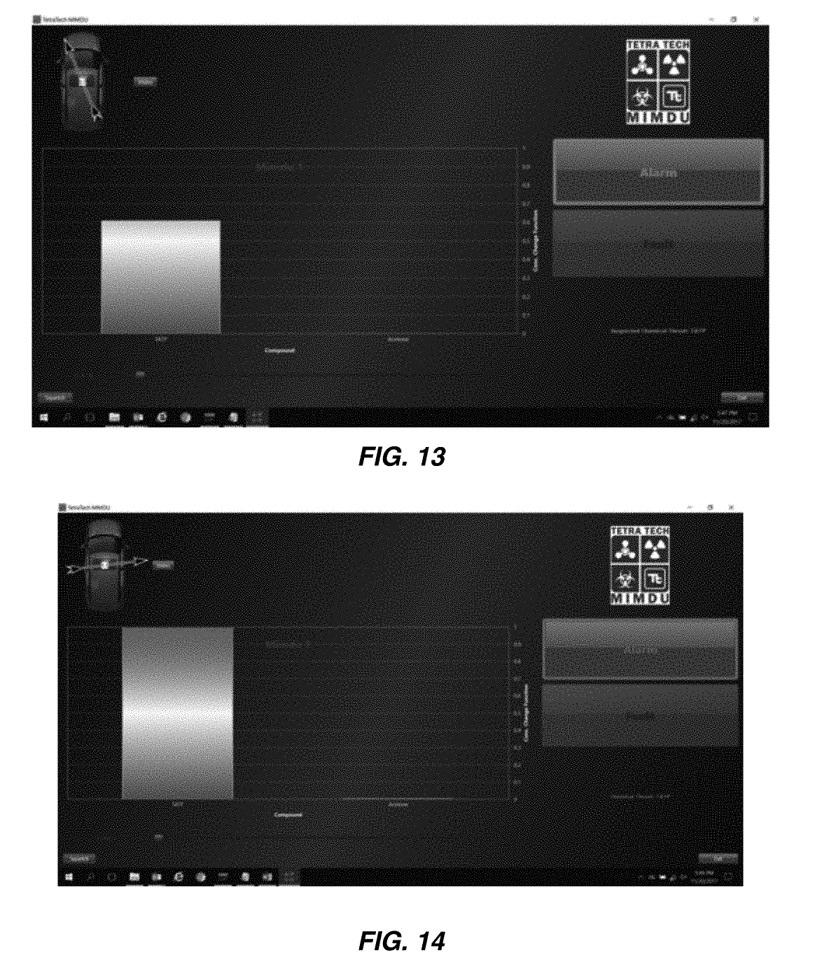

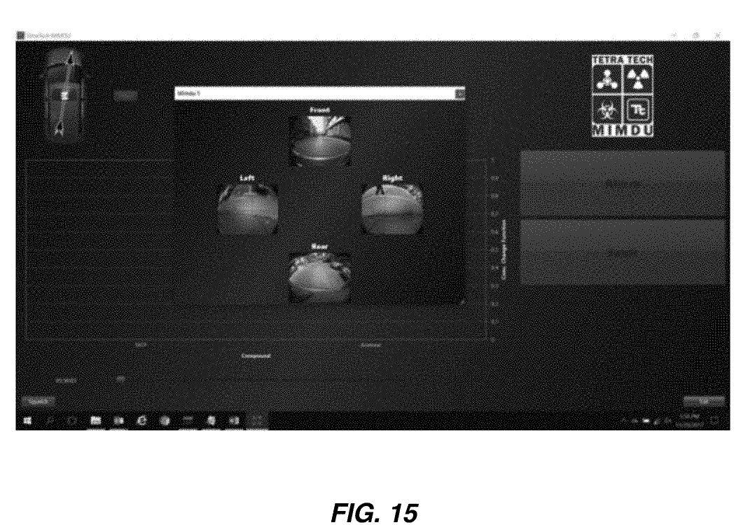

FIGS. 13-15 show an exemplary screenshot of the graphical user interface 20. The graphical user interface 20 can include views from one or more cameras 30 of the system 10, relative concentrations of detected chemicals of interest, a system status of the multiple subsystems, GPS location data from a GPS device as shown in FIG. 12, and weather substation 142 information (e.g., wind direction as shown by the plan image of a vehicle with an arrow superimposed thereon). FIG. 13 illustrates an initial detection of a substance of interest and graphically displays information regarding the detected substance, such as a concentration of the substance. FIG. 14 shows a graphical representation of an alarm condition with a higher concentration detected. FIG. 15 illustrates the graphical user interface including views of the cameras 30 of the system 10. Preferably, during an alarm condition, raw data is archived from the various subsystems including weather and GPS information, and the data is time stamped along with images recorded on the cameras 30. An alarm condition is displayed on the graphical user interface 20 for immediate evaluation, preferably including an audible alarm signal. An operator can then select the displayed alarm condition in order to view more detailed data sets to confirm the alarm condition. The operator can then adjudicate the alarm condition, deciding whether to save the full data set on the archiving device or server, or instead to delete the data and enter a note into the official record documenting that it was a nuisance alarm or some other known acceptable condition. Real time GPS data and/or weather data are used to provide instant feedback to a driver of the mobile vehicle 12, if the driver desires to track a chemical plume, or continuously bisect a plume to find its source.

### Command and Control Communications Subsystem

The command and control communications subsystem 28 incorporates an integrated command and control communications system capable of transmitting event data and associated voice traffic thru encrypted channels. This command and control feature capability, combined with video data from one or more integrated cameras 30 and optical character recognition and facial recognition software, can be used to direct the immediate response of police, border security, customs, or intelligence agents in the field to a specific location. The combined data set can be used to prosecute those individuals caught performing illegal acts.

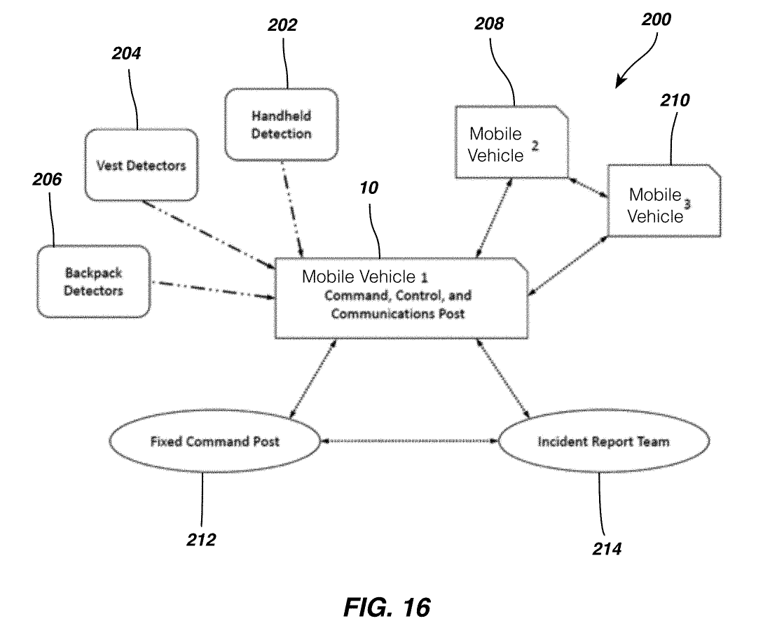

Referring to FIG. 16, the command and control communications subsystem 28 enables the sensing system 10 to function as part of a larger command and control group 200. The sensing system 10 may be in electronic communication with one or more handheld detectors 202, vest detectors 204 worn by personnel on the ground, and backpack detectors 206. The sensing system 10 may also be in communication with additional mobile vehicles 208 and 210, a fixed command post 212, and/or an incident report team 214. Command and control features of embodiments of the present disclosure enable an operator of the sensing system 10 to direct and control actions of a response force capable of interdicting contraband. Global positioning system modules may be included on the handheld detectors 202, vest detectors 204, and backpack detectors 206 such that an operator may identify a location of each individual using a detector relative to the sensing system 10, and to further measure activity on the various remote detectors.

Referring to FIG. 16, real time communications are permitted between the mobile vehicle 12 driver and the system 10 operator, as well as communications to a fixed command post. The system also permits communications with incident response personnel, other patrolmen, or other mobile vehicles with the same or similar features on board. Data from all of these potential data sources can be tagged with GPS location information and funneled to the fixed command post where all data can be monitored and analyzed in one fixed location.

Preferably, the command and control communications subsystem mirrors data from the system 10 operator software and archiving device(s) and/or server(s) to another location away from the van (usually a fixed command post) where the data can be further analyzed for trends or anomalies, and then be archived for various purposes.

The intended concepts for operation of the system can be summarized in the following scenarios:
Portal Mode: The system 10 (vehicle with integrated detectors and subsystems) is kept stationary temporarily. Vehicles or pedestrians are funneled to pass by the system vehicle 12 with available pedestrian and traffic control barriers (not included with the system). In the portal mode, when the radar sensor 114 detects an object within proximity to the mobile vehicle 12, the system 10 may record data during a designated time window surrounding detection of an object within proximity to the mobile vehicle 12.
Checkpoint Mode: The system vehicle 12 is kept stationary temporarily. Drivers of vehicles are intentionally stopped in the proximity of the vehicle 12 (security or identification/passport check). One or more operators are stationed outside the system vehicle 12 to direct an extension hose inside each vehicle, either inconspicuously or conspicuously.
Search Mode: The system 10 is relocated from container to container, airplane to airplane, or other container type to other container type. One or more operators outside the system vehicle 12 use an extension hose to draw air out of the vehicles, shipping containers, buildings, or other structures outside the system vehicle 12.
Survey Mode: The system 10 is operated in a fully mobile mode with one or more operators seated inside the system vehicle 12. All detectors are simultaneously operational. Any radioactive material or chemical/biological compound within the vicinity of the system vehicle 12 is detected. Additionally or alternatively, environmental concentrations of environmental hazards can be detected and traced back to their source.

Novel features of the system 10 are primarily the integration of disparate wholly passive detector systems including radiological detection and chemical/biological detection, a single robust user interface to operate all detector systems simultaneously, mobility of previously fixed laboratory-only equipment, customization, and response force command and control communications systems.

There are no other real-time in situ wholly passive mobile systems on the market today that combine chemical, biological, radiological, nuclear, and explosives detection in a single, easy to deploy and operate system. The available individual detector technologies typically require large pieces of equipment, are not hardened for mobile use, and require detailed scientific knowledge to decipher the output. Configuring these previously stand-alone systems into smaller hardened packages installed into a mobile unit and providing a simple user interface provides a new way of using these detectors that can greatly impact multiple industries and organizations.

The mobility of the system 10 allows large areas to be surveyed for long periods of time. Similar methods of detection, such as bomb or drug sniffing dogs, are limited in both supply and mobility. This can be due to the specific material training for each dog, shortage of trained dogs across the wide range of illicit material for detection assignment, short work times, inherent animal fatigue, death in the case of certain drugs, and the difficulty in using dogs in a moving mobile unit.

The system 10 described herein is normally set up to detect and identify anything present in the vicinity of the system vehicle 12. Alternatively, the system 10 can be optimized for detection of specific chemicals, biological agents, and/or radioisotopes. Comparatively, similar methods of detection are trained dogs, which are trained only for drugs or a specific chemical compound family. The ability of the system 10 to detect and identify specific drugs, explosives, or environmental pollutants all at the same time, particularly while moving, is a unique benefit over current methods.

The simple user interface described herein for the data output of the radiation subsystem 14 and the chemical/biological subsystem 16 is unique to most chemical/biological sampling systems because it allows for the operation of the entire system, including multi-modality detection, with limited training. Alarm features that are integrated with video recording allow a user to keep visual information tied to the conditions surrounding the system vehicle for future use. Other systems used for these purposes do not provide simple operation. In the case of dogs, the handlers required rigorous training in order to interpret the dog's reaction. In the case of scientific instrumentation, the operators require years of study to understand interpretation of the output from such devices.

The foregoing description of preferred embodiments of the present disclosure has been presented for purposes of illustration and description. The described preferred embodiments are not intended to be exhaustive or to limit the scope of the disclosure to the precise form(s) disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the concepts revealed in the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

What is claimed is:

1. A mobile multi-modality passive sensing system comprising:
a mobile vehicle;
a passive radiological detection device mounted in the mobile vehicle for detecting potentially harmful radiological materials proximate to the mobile vehicle;
a passive chemical detection device mounted in the mobile vehicle for detecting potentially harmful chemical materials proximate to the mobile vehicle;
a user interface device located in the mobile vehicle;
a central processing device located in the mobile vehicle wherein the central processing device is in communication with the passive radiological detection device, the passive chemical detection device, and the user interface device, and wherein the central processing device:
receives data from (1) the passive radiological detection device related to potentially harmful radiological materials proximate to the mobile vehicle and (2) the passive chemical detection device related to potentially harmful chemical materials proximate to the mobile vehicle;
processes the data resulting with processed data; and
sends the processed data to the user interface device to be displayed on the user interface device.

2. The mobile multi-modality passive sensing system of claim 1, further comprising an air sampling subsystem comprising a tube connected to the chemical detection device and a vacuum pump for drawing in air wherein air is drawn into the tube from outside the mobile vehicle and directed to the chemical detection device for real time analysis.

3. A mobile multi-modality passive sensing system comprising:
a mobile vehicle;
a passive radiological detection device mounted in the mobile vehicle for detecting potentially harmful radiological materials proximate to the mobile vehicle;
a passive chemical detection device mounted in the mobile vehicle for detecting potentially harmful chemical materials proximate to the mobile vehicle;
a computing device comprising a processor and a user interface device, the computing device located in the mobile vehicle in communication with the radiological detection device and the chemical detection device wherein data from the radiological detection device and the chemical detection device is sent to the computing device for processing and then to the user interface device to be displayed to a user.

4. The mobile multi-modality passive sensing system of claim 3 further comprising an air sampling subsystem comprising a tube connected to the chemical detection device and a vacuum pump for drawing in air wherein air is drawn into the tube from outside the mobile vehicle and directed to the chemical detection device for real time analysis.

5. The mobile multi-modality passive sensing system of claim 4 further comprising a weather substation in communication with the computing device for gathering weather data and relaying such weather data to the computing device.

6. The mobile multi-modality passive sensing system of claim 4 further comprising a GPS device in communication with the computing device for providing GPS location data to the computing device.

7. The mobile multi-modality passive sensing system of claim 5 further comprising a computer readable storage device for archiving data originating from the radiological detection device, the chemical detection device, and the weather substation.

8. The mobile multi-modality passive sensing system of claim 6 further comprising a computer readable storage device for archiving data originating from the radiological detection device, the chemical detection device, and the GPS device.

9. The mobile multi-modality passive sensing system of claim 4 wherein the mobile vehicle further comprises a window and at least one camera directed toward the window for collecting images gathered through the window, and wherein the at least one camera is in communication with the computing device.

10. The mobile multi-modality passive sensing system of claim 5 further comprising:
a GPS device in communication with the computing device for providing GPS location data to the computing device; and
a computer readable storage device for archiving data originating from the radiological detection device, the chemical detection device, the weather substation and the GPS device.

11. The mobile multi-modality passive sensing system of claim 10 wherein the mobile vehicle further comprises a window and a camera directed toward the window for collecting images gathered through the window, wherein the camera is in communication with the computing device, and wherein the computer readable storage device archives data originating from the radiological detection device, the chemical detection device, the weather substation, the GPS device, and the at least one camera.

12. The mobile multi-modality passive sensing system of claim 4 further comprising a command and control communications subsystem comprising a member selected from the group consisting of a handheld detector, a vest detector, a backpack detector, one or more additional mobile vehicles, and any combinations thereof.

13. The mobile multi-modality passive sensing system of claim 11 further comprising a command and control communications subsystem comprising a member selected from the group consisting of a handheld detector, a vest detector, a backpack detector, one or more additional mobile vehicles, and any combinations thereof.

14. A method of passively monitoring an environment for potential chemical dangers and potential radiological dangers using a mobile vehicle, the method comprising the steps of:
(i) operating a passive radiological detection device on a mobile vehicle to acquire radiological data;
(ii) operating a passive chemical detection device on the mobile vehicle to acquire chemical data;
(iii) sending the radiological data to a computing device;
(iv) sending the chemical data to the computing device;
(v) processing the radiological data;
(vi) processing the chemical data;
(vii) sending the processed radiological data and the processed chemical data to a display device for displaying the processed radiological data together with the processed chemical data.

15. The method of claim 14 further comprising the step of producing an alarm condition signal to be displayed on the display device if the radiological data or the chemical data exceed a minimum concentration threshold.

16. The method of claim 15 further comprising the step of archiving the radiological data if the radiological data exceed a minimum concentration threshold.

17. The method of claim 15 further comprising the step of archiving the chemical data if the chemical data exceed a minimum concentration threshold.

18. The method of claim 15 further comprising the step of tagging the radiological data with GPS coordinates of the mobile vehicle in response to the alarm condition signal.

19. The method of claim 15 further comprising the step of tagging the chemical data with GPS coordinates of the mobile vehicle in response to the alarm condition signal.

20. The method of claim 15 further comprising the step of archiving video data gathered from a camera mounted on the mobile vehicle in response to the alarm condition signal.
